(19) United States
(12) Patent Application Publication
(10) Pub. No.: US 2019/0029951 A1
(43) Pub. Date: Jan. 31, 2019

(54) EXTENDED RELEASE FORMULATIONS OF VELIPARIB FOR THE TREATMENT OF CANCER

(71) Applicant: AbbVie Inc., North Chicago, IL (US)

(72) Inventors: Hao Xiong, North Chicago, IL (US); Alyssa E. Debord, Grayslake, IL (US); Philip Komarnitsky, Cambridge, MA (US); Silpa Nuthalapati, North Chicago, IL (US); Sven Stodtmann, Ludwigshafen (DE)

(21) Appl. No.: 16/073,042

(22) PCT Filed: Jan. 31, 2017
(86) PCT No.: PCT/US2017/015792

§ 371 Date: Jul. 26, 2018

Related U.S. Application Data
(60) Provisional application No. 62/289,808, filed on Feb. 1, 2016.

(51) Int. Cl. A61K 9/00; A61K 31/4184; A61P 35/00

(52) U.S. Cl. CPC A61K 9/0002; A61P 35/00; A61K 31/4184; A61K 9/0053

(57) ABSTRACT

Described herein are extended release formulations of veliparib, ways to make them, and methods of treating cancer using them.

### CROSS-REFERENCE TO RELATED APPLICATIONS

This application claims priority to U.S. Provisional Application Ser. No. 62/289,808, filed Feb. 1, 2016, which is incorporated by reference in its entirety.

### FIELD OF THE INVENTION

This invention pertains to extended release (ER) formulations of veliparib, and methods of treating cancer using them.

### BACKGROUND OF THE INVENTION

Poly(ADP-ribose)polymerase (PARP) or poly(ADP-ribose)synthase (PARS) is a nuclear enzyme that has an essential role in recognizing DNA damage, facilitating DNA repair, controlling RNA transcription, mediating cell death, and regulating immune response. PARP activity is required for the repair of single-stranded DNA breaks through the base excision repair pathways. Cancer cells are often deficient in double-stranded DNA-repair capability, and are therefore more dependent on PARP directed single-stranded DNA-repair than are normal cells. Consequently, inhibition of PARP enhances the anti-tumor effects of DNA-damaging agents in many cancer cells.

Veliparib (ABT-888, 2-[(2R)-2-methylpyrrolidin-2-yl]-1H-benzimidazole-4-carboxamide) is an orally bioavailable PARP inhibitor. Monotherapy trials with an immediate release formulation of veliparib showed antitumor activity in BRCA+ cancers. However, due to the dose and dosing frequency needed for monotherapy efficacy (200-400 mg BID) of veliparib, and the side effects some patients have experienced while treated with veliparib, an improved dosage form, for example, an extended release formulation is desirable.

### SUMMARY OF THE INVENTION

It has been discovered that the extended release (ER) formulations of veliparib were found to unexpectedly exhibit increased efficacy compared to the immediate release (IR) formulation. Maximum concentrations (Cₘₐₓ) of veliparib in plasma for the ER formulations were statistically significantly lower than the IR formulation, while the area under the plasma concentration-time curve (AUC) and the minimum plasma concentration were maintained. Higher minimal concentration was achieved with ER formulation compared to IR formulation under twice-daily dosing schedule. The plasma concentration profile observed for the ER formulations resulted in comparable or less adverse events and an unexpected improvement tumor response when compared to the corresponding IR formulation.

One aspect pertains to an extended release (ER) oral pharmaceutical formulation of veliparib, or a pharmaceutically acceptable salt thereof.

Another aspect pertains to an extended release (ER) oral pharmaceutical formulation of veliparib, or a pharmaceutically acceptable salt thereof, wherein the dose-normalized C₂₄ (concentration at 24 hours post dosing) of veliparib after once-daily administration of the formulation to a human subject is between about 1 and about 2 ng/mL/mg. Another aspect pertains to an extended release (ER) oral pharmaceutical formulation of veliparib, or a pharmaceutically acceptable salt thereof, wherein the dose-normalized C₁₂ (concentration at 12 hours post dosing) of veliparib after twice-daily administration of the formulation to a human subject is between about 3 and about 5 ng/mL/mg.

Another aspect pertains to an extended release (ER) oral pharmaceutical formulation of veliparib, or a pharmaceutically acceptable salt thereof, wherein the peak-to-trough concentration ratio is about 2 following twice-daily dosing to a human subject. Another aspect pertains to an extended release (ER) oral pharmaceutical formulation of veliparib, or a pharmaceutically acceptable salt thereof, wherein the peak-to-trough concentration ratio is about 4 following once-daily dosing to a human subject.

One aspect pertains to a method of treating cancer comprising administering a therapeutically effective amount of an extended release (ER) oral pharmaceutical formulation of veliparib, or a pharmaceutically acceptable salt thereof.

### BRIEF DESCRIPTION OF THE DRAWINGS

FIG. 1 shows veliparib mean plasma concentration-time profiles for a single dose of extended release or immediate release formulations under fasting conditions. Mean (+Standard Deviation) Plasma Concentration versus Time Profiles of Veliparib After a Single Dose of Veliparib in an ER or IR Formulation under Fasting Conditions in Subjects with Solid Tumors.

FIG. 2 shows best tumor response in ovarian cancer by BRCA status.

FIG. 3 shows Best tumor response in patients with breast cancer (all with BRCA mutation).

FIG. 4 shows exposure-response model for tumor growth inhibition by veliparib.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. In this application, the use of “or” means “and/or” unless stated otherwise. Furthermore, the use of the term “including”, as well as other forms, such as “includes” and “included”, is not limiting. With reference to the use of the words “comprise” or “comprises” or “comprising” or “consisting essentially of” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The term “about” as used herein, means approximately, and in most cases within 10% of the stated value.

The terms “treat”, “treating” and “treatment” refer to a method of alleviating or abrogating a disease and/or its attendant symptoms.

The term “subject” is defined herein to include animals such as mammals, including, but not limited to, primates (e.g., humans), cows, sheep, goats, horses, dogs, cats, rabbits, rats, mice and the like. In one embodiment, the subject is a human.

The terms “patient” and “subject” are used herein interchangeably.

The term “therapeutically effective amount” refers to the amount sufficient to induce a desired biological, pharmacological, or therapeutic outcome in a subject. A therapeutically effective amount of a compound can be employed as a zwitterion or as a pharmaceutically acceptable salt. A therapeutically effective amount means a sufficient amount of the compound to treat or prevent a disease or disorder ameliorated by a PARP inhibitor at a reasonable benefit/risk ratio applicable to any medical treatment.

The terms “pharmaceutically acceptable acid addition salt” and “pharmaceutically acceptable salt”, which are used interchangeably herein, refer to those salts in which the anion does not contribute significantly to the toxicity or pharmacological activity of the salt, and, as such, they are the pharmacological equivalent of the base form of the active agent.

The term “Cₘₐₓ” as used herein, means maximum observed plasma concentration of veliparib, produced by the ingestion of the composition of the invention or the IR comparator.

The term “Cₘᵢₙ” as used herein, means minimum plasma concentration of vehparb, produced by the ingestion of the composition of the invention or the IR comparator.

The term “C₁₂” as used herein, means the concentration of veliparib at 12 hours post dose administration under a twice-daily dosing regimen.

The term “C₂₄” as used herein, means the concentration of veliparib at 24 hours post dose administration under a once-daily dosing regimen.

The term “Tₘₐₓ” as used herein, means time to the maximum observed plasma concentration of veliparib.

The term “AUC” as used herein, means area under the plasma concentration-time curve.

The term “AUC_{INF}” as used herein, means area under the plasma concentration-time curve from time zero to infinity

The term “BRCA-wt” as used herein, means basal-like breast cancer.

The term. “BRCA-mut” means a mutation in the Breast Cancer 1 or Breast Cancer 2 gene.

The term “peak-to-trough ratio” as used herein, is expressed as

The term “pharmaceutically acceptable” as used herein, means those compounds which are, within the scope of sound medical judgment, suitable for use in contact with the tissues of humans and lower animals without undue toxicity, irritation, allergic response, and the like, in keeping with reasonable bereft/risk ratio, and effective for their intended use.

The term “efficacy” as used herein refers to an inhibitory effect on tumor growth, which can be manifested as slowing, retarding, arresting or even reversing (i.e., shrinking) growth of the tumor. In some embodiments, the effect on tumor growth is associated with suppression by a PARP inhibitory compound, for example Veliparib. In clinical sittings, efficacy can be measured by one skilled in the art and by a number of efficacy endpoints, for example, overall survival (OS), progression free survival (PPS), time to progression (TTP), time to treatment failure (TTF), event-free survival (EFS), time to next treatment (TINT), objective response rate (ORR), duration of response (DoR), and/or response rate, for example, partial response (PR) or complete response (CR). The term “partial response” or “PR” as used herein, means a partial response of the target lesion, indicated by at least a 30% decrease in the sum of diameters of target lesions, taking as reference the baseline sum diameters.

The term “adverse effects” as used herein, means those physiological effects to various systems in the body such as cardiovascular systems, nervous system, digestive system, and body as a whole, which cause pain and/or discomfort to the individual subject.

The term “trace” as used herein, means a small but measureable amount, typically <0.5 w/w %.

The term “ER” as used herein, means extended release.

The term “extended release” as used herein, refers to a composition in which a drug is released over an extended period of time, such as over 2, 4, 6, 10, 12, 15, 18, 20 or 24 hours.

The term “IR” as used herein, means immediate release.

The term “BID” or “twice-daily” as used herein, means twice during each 24 hour period.

The term “QD” or “once-daily” as used herein, means once during each 24 hour period.

### Methods of Treatment

One embodiment pertains to an extended release (ER) oral pharmaceutical formulation of a PARP inhibitor. Another embodiment pertains to an extended release (ER) oral pharmaceutical formulation of veliparib.

One embodiment pertains to an extended release (ER) oral pharmaceutical formulation of veliparib, wherein the dose-normalized C24 (concentration at 24 hours post dosing) of veliparib after once-daily administration of the formulation to a human subject is between about 1 and about 2 ng/mL/mg.

Another embodiment pertains to an extended release (ER) oral pharmaceutical formulation of veliparib, wherein the dose-normalized Cu (concentration at 12 hours post dosing) of veliparib after twice-daily administration of the formulation to a human subject is between about 3 and about 5 ng/mL/mg.

Another embodiment pertains to an extended release (ER) oral pharmaceutical formulation of veliparib, wherein the peak-to-trough concentration ratio is about 2 following twice-daily dosing to a human subject. Another embodiment pertains to an extended release (ER) oral pharmaceutical formulation of veliparib, wherein the peak-to-trough concentration ratio is about 4 following once-daily dosing to a human subject.

One embodiment pertains to a method of treating cancer comprising administering a therapeutically effective amount of an extended release (ER) oral pharmaceutical formulation of a PARP inhibitor. Another embodiment pertains to a method of treating cancer comprising administering a therapeutically effective amount of an extended release (ER) oral pharmaceutical formulation of veliparib.

One embodiment pertains to a method of treating cancer comprising administering a therapeutically effective amount of an extended release (ER) oral pharmaceutical formulation of a PARP inhibitor, or a pharmaceutically acceptable salt thereof, wherein efficacy of the cancer treatment is increased by at least 50%, at least 100%, at least 200% or at least 300% as compared to an equivalent amount of an immediate release formulation of the PARP inhibitor. Another embodiment pertains to a method of treating cancer comprising administering a therapeutically effective amount of an extended release (ER) oral pharmaceutical formulation of a PARP inhibitor, or a pharmaceutically acceptable salt thereof, wherein efficacy of the cancer treatment is increased by at least 50%, at least 100%, at least 200% or at least 300% as compared to an equivalent amount of an immediate release formulation of the PARP inhibitor in an otherwise identical dosage regimen.

One embodiment pertains to a method of treating cancer comprising administering a therapeutically effective amount of an extended release (ER) oral pharmaceutical formulation of veliparib, or a pharmaceutically acceptable salt thereof, wherein efficacy of the cancer treatment is increased by at least 50%, at least 100%, at least 200% or at least 300% as compared to an equivalent amount of an immediate release formulation of Veliparib. One embodiment pertains to a method of treating cancer comprising administering a therapeutically effective amount of an extended release (ER) oral pharmaceutical formulation of veliparib, or a pharmaceutically acceptable salt thereof, wherein efficacy of the cancer treatment is increased by at least 50%, at least 100%, at least 200% or at least 300% as compared to an equivalent amount of an immediate release formulation of Veliparib in an otherwise identical dosage regimen.

One embodiment pertains to a method of treating cancer comprising administering a therapeutically effective amount of an extended release (ER) oral pharmaceutical formulation of veliparib, wherein the AUC of the plasma concentration profile of veliparib after administration of the formulation to a human subject is between about 5000 and about 20000 ng hr/mL.

One embodiment pertains to a method of treating cancer comprising administering a therapeutically effective amount of an extended release (ER) oral pharmaceutical formulation of veliparib, wherein the dose-normalized C₂₄ (concentration at 24 hours post dosing) of veliparib after once-daily administration of the formulation to a human subject is between about 1 and about 2 ng/mL/mg.

Another embodiment pertains to a method of treating cancer comprising administering a therapeutically effective amount of an extended release (ER) oral pharmaceutical formulation of veliparib, wherein the dose-normalized Cu (concentration at 12 hours post dosing) of veliparib after twice-daily administration of the formulation to a human subject is between about 3 and about 5 ng/mL/mg.

Another embodiment pertains to a method of treating cancer comprising administering a therapeutically effective amount of an extended release (ER) oral pharmaceutical formulation of veliparib, wherein the peak-to-trough concentration ratio is about 2 following twice-daily dosing.

Another embodiment pertains to a method of treating cancer comprising administering a therapeutically effective amount of an extended release (ER) oral pharmaceutical formulation of veliparib, wherein the peak-to-trough concentration ratio is about 4 following once-daily dosing.

In one embodiment, the release period is from about 2 to about 24 hours. In another embodiment, the release period is from about 6 to about 16 hours. In another embodiment, the release period is about 6 hours. In another embodiment, the release period is about 12 hours. In another embodiment, the release period is about 16 hours.

In one embodiment, the amount of veliparib in the formulation is about 50 mg to about 1000 mg. In another embodiment, the amount of veliparib in the formulation is about 50 mg to about 500 mg. In another embodiment, the amount of veliparib in the formulation is about 100 mg to about 800 mg. In another embodiment, the amount of veliparib in the formulation is about 100 mg to about 400 mg. In another embodiment, the amount of veliparib in the formulation is about 200 mg.

In one embodiment, the cancer is selected from the group consisting of breast, ovarian, fallopian tube, primary peritoneal, and lung. In another embodiment, the cancer is lung cancer. In another embodiment, the cancer is non-small cell lung cancer. In another embodiment, the cancer is squamous non-small cell lung cancer. In another embodiment, the cancer is non-squamous non-small cell lung cancer. In another embodiment, the cancer is ovarian cancer. In another embodiment, the cancer is BRCA deficient breast cancer.

### Experimental Studies

Eligible patients had metastatic BRCA+ cancer, BRCA± high-grade serious ovarian/primary peritoneal/fallopian tube cancer (OC), (part 1/2), or BRCA+ breast cancer (BC), or OC (part 3).

Part 1—Pharmacokinetic (PK) assessment: patients received single dose of extended release veliparib (ER-v) (3 different ER-v's tested) or immediate release veliparib (IR-v) 200 mg in fed/fasted state on days 1/3/5 (3-period crossover). Upon conclusion of assessments and procedures in Period 3, subjects were allowed to continue veliparib monotherapy at 300 mg BID with an option to escalate to 400 mg BID at the discretion of the Investigator using IR-v.

Part 2—3+3 dose escalation: patients received ER-v daily (QD or BID) starting at 200 mg. Upon conclusion of the DLT assessment period (first cycle of dosing), patients were allowed to, beginning on Cycle 2 Day 1, continue treatment with ER-v at the dose level which they received and tolerated or may change to IR-v monotherapy at 300 mg BID with escalation to 400 mg BID at the discretion of the Investigator.

Part 3—safety expansion: ER-v was administered continuously at the recommended phase II dose (RP2D).

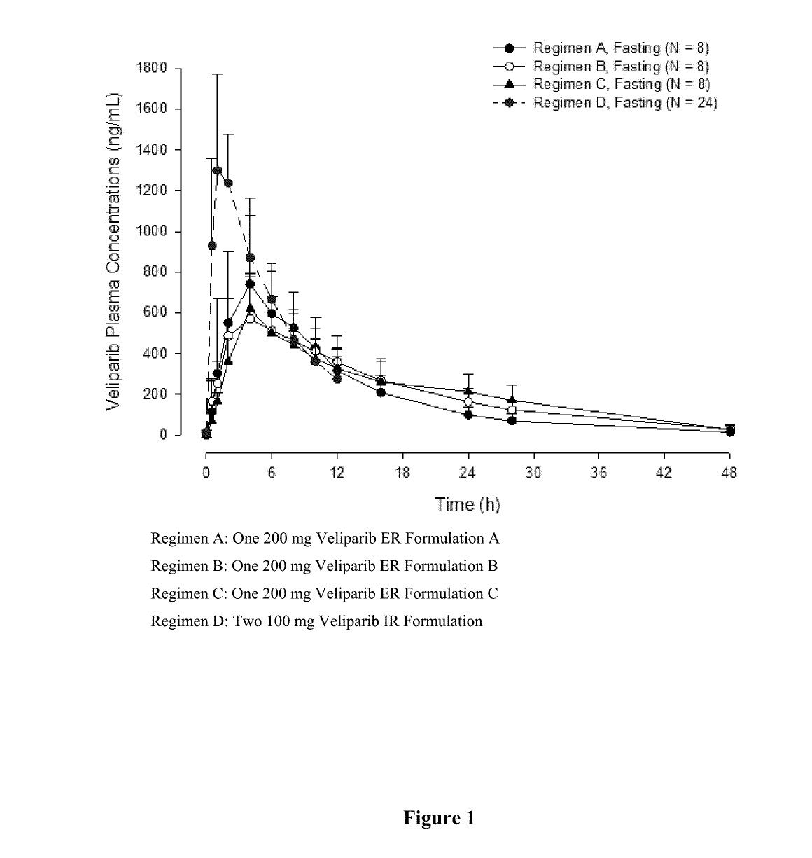

Seventy-one patients (96% female, 75% OC) received veliparib (part 1/2/3, n=24/35/12). Patients were heavily pretreated and included OC patients refractory to platinum. After a single dose, ER-v had a longer Tₘₐₓ(3.5 to 5.0 vs 1.5 hours) and lower Cₘₐₓ (629 to 757 vs 1429 ng/mL) than IR-v; and AUC_{∞} was similar (FIG. 1, Error! Reference source not found. and Error! Reference source not found.). Food intake moderately increased Cₘₐₓ and Tₘₐₓ (Error! Reference source not found.1 and Error! Reference source not found.). Under QD dosing, the median peak-to-trough concentration ratio was about 4 (Error! Reference source not found.). Under BID dosing, the median peak-to-trough concentration ratio was about 2 (Error! Reference source not found.4). Three patients experienced dose limiting toxicity (DLT): ⅖ patients treated at 600 mg BID ER-v (G2 asthenia and G3 nausea and vomiting) and ⅓ pts treated at 800 mg QD ER-v (G3 seizure). The ER-v RP2D was determined to be 400 mg BID. Grade ¾ treatment-emergent adverse event (TEAE) (part 1/2/3) occurred in 25/20/17% of patients; most common were thrombocytopenia (8/6/0%), small intestinal obstruction (4/0/8%), nausea (0/3/8%), and vomiting (0/3/8%). Confirmed and unconfirmed responses were observed in patients with BC (6/16, 38%) and OC (8/44, 18%). The rate of Grade 2 and 3 nausea in the Part 3 (ER-v only) of the trial was 25% (3/12 subjects).

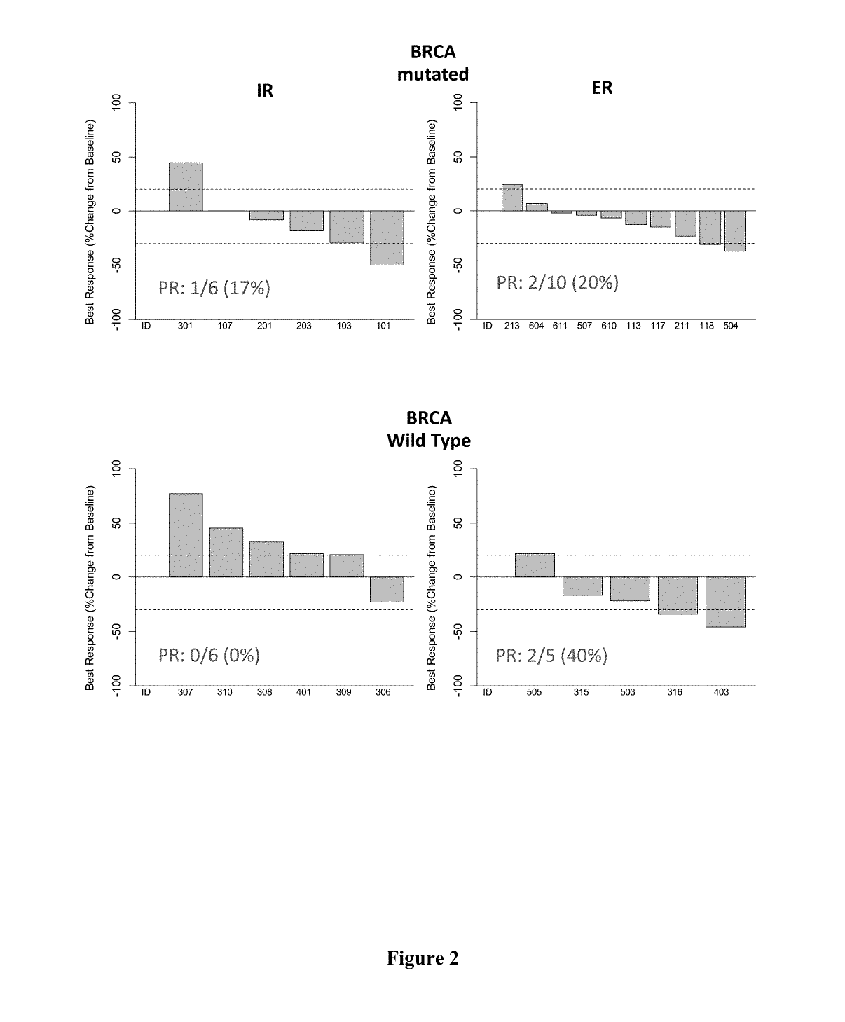

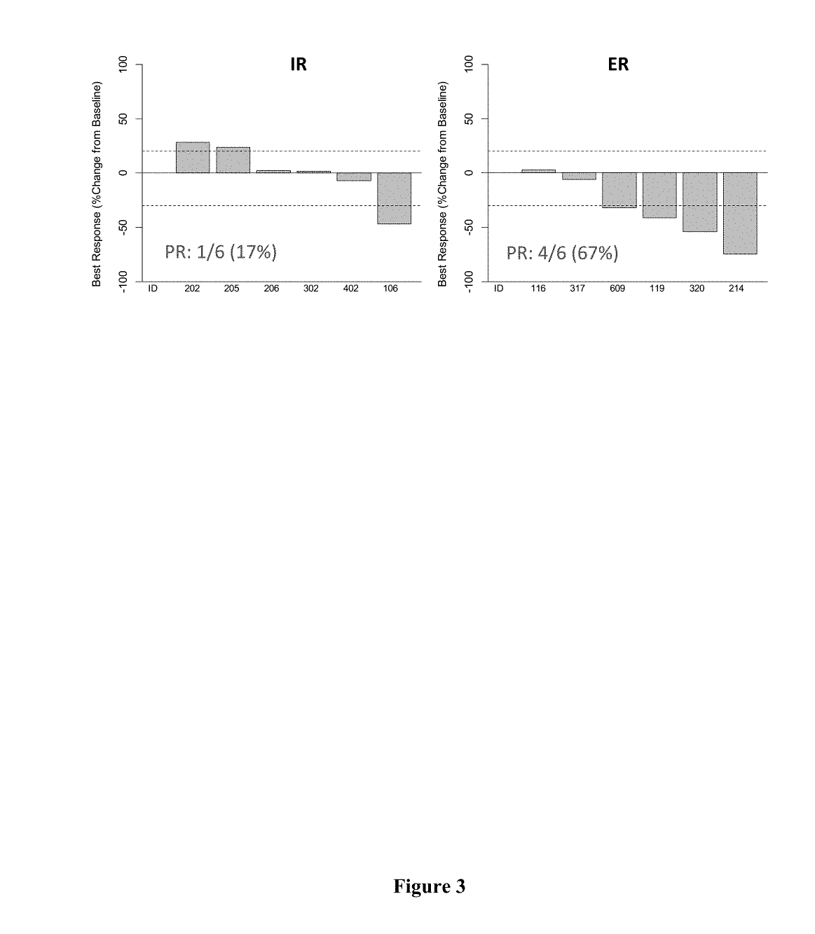

Best tumor responses were compared between patients from Part 1 who received IR-v as the primary treatment and patients in Part 2 and Part 3 of the study who received ER-v formulation C. Two single doses of veliparib in ER formulations at 200 mg dose were deemed to have negligible effect on tumor response in patients enrolled in Part 1. Patients in Part 2 whose partial response (PR) were observed after switching from ER-v to IR-v were excluded from the analysis due to the ambiguous attribution to the formulation effect. In patients with ovarian cancer and BRCA mutation, 40% (⅖) achieved PR under ER-v treatment as compared to 0% (0/6) under IR-v treatment (FIG. 2). In patients with breast cancer and BRCA mutation, 67% (4/6) achieved PR under ER-v treatment as compared to 17% (⅙) under IR-v treatment (FIG. 3 and Table 5).

TABLE 1
Veliparib Pharmacokinetic Parameters (Mean ± SD) Following
A Single Oral Administration of A Single Dose of Veliparib
Extended Release or Immediate Release in Cancer Patients

| Pharma- | Extended | Extended | Extended | |
|---|---|---|---|---|
| cokinetic | Release | Release | Release | |
| Parameter | Formu- | Formu- | Formu- | Immediate |
| (Units) | lation A | lation B | lation C | Release |
| N | 8 | 8 | 8 | 24 |
| | 200 mg Veliparib, Fasting | | | |
| Cₘₐₓ (ng/mL) | 757 ± 316 | 643 ± 161 | 629 ± 147 | 1429 ± 385 |
| Tₘₐₓ (h) | 4.5 ± 0.9 | 5.0 ± 2.8 | 3.5 ± 0.9 | 1.5 ± 0.8 |
| t_{1/2} (hr)^{a} | 7.4 ± 1.8 | 8.6 ± 1.7 | 8.2 ± 2.9 | 4.6 ± 1.5 |
| AUCₜ | 9.44 ± 3.17 | 10.3 ± 2.83 | 10.5 ± 3.10 | 8.25 ± 1.83 |
| (μg · h/mL) | | | | |
| AUC_{INF} | 9.65 ± 3.21 | 10.7 ± 3.07 | 11.4 ± 3.22 | 10.6 ± 3.05 |
| (μg · h/mL) | | | | |
| | 200 mg Veliparib, Nonfasting | | | |
| Cₘₐₓ (ng/mL) | 1012 ± 435 | 881 ± 232 | 885 ± 126 | NA |
| Tₘₐₓ (h) | 5.3 ± 1.5 | 7.0 ± 1.9 | 6.8 ± 1.8 | NA |
| t_{1/2} (hr)^{a} | 6.6 ± 1.4 | 6.8 ± 3.1 | 6.4 ± 2.5 | NA |
| AUCₜ | 10.0 ± 2.94 | 10.6 ± 2.46 | 12.2 ± 2.20 | NA |
| (μg · h/mL) | | | | |
| AUC_{INF} | 10.2 ± 3.00 | 10.8 ± 2.48 | 12.4 ± 2.18 | NA |
| (μg · h/mL) | | | | |

^{a}Harmonic mean and pseudo-standard deviation.

TABLE 2
Relative Bioavailability and 90% Confidence Intervals for the Assessment
of Extended Release Versus Immediate Release Formulation and the
Effect of Food on the Extended Release Formulation

| | | Relative Bioavailability | |
|---|---|---|---|
| Regimens | Pharmacokinetic | Point | 90% Confidence |
| Test vs. Reference | Parameter | Estimate | Interval |
| ER-A Fasting | Cₘₐₓ | 0.541 | 0.454-0.646 |
| vs. IR Fasting | AUC_{INF} | 1.007 | 0.923-1.099 |
| ER-B Fasting | Cₘₐₓ | 0.448 | 0.378-0.531 |
| vs. IR Fasting | AUC_{INF} | 1.040 | 0.988-1.095 |
| ER-C Fasting | Cₘₐₓ | 0.420 | 0.372-0.475 |
| vs. IR Fasting | AUC_{INF} | 0.966 | 0.871-1.071 |
| ER-A Nonfasting | Cₘₐₓ | 1.350 | 1.131-1.612 |
| vs. Fasting | AUC_{INF} | 1.060 | 0.972-1.157 |
| ER-B Nonfasting | Cₘₐₓ | 1.377 | 1.161-1.633 |
| vs. Fasting | AUC_{INF} | 1.020 | 0.969-1.074 |
| ER-C Nonfasting | Cₘₐₓ | 1.424 | 1.261-1.609 |
| vs. Fasting | AUC_{INF} | 1.105 | 0.996-1.225 |

TABLE 3
Veliparib Pharmacokinetic Parameters (Mean ±
SD) on Day 3 Following Once-Daily (QD) Oral Administration
of Veliparib Extended Release Formulation C

| Pharma- | | | | |
|---|---|---|---|---|
| cokinetic | 200 mg | 400 mg | 600 mg | 800 mg |
| Parameters | (N = 4) | (N = 4) | (N = 6) | (N = 2) |
| Tₘₐₓ (h) | 4.5 ± 3 | 5.5 ± 1.9 | 4.5 ± 2.7 | 5.0 |
| Cₘₐₓ (ng/mL) | 832 ± 50 | 1800 ± 492 | 3352 ± 1229 | 4865 |
| C₂₄ (ng/mL) | 208 ± 89 | 476 ± 291 | 2111 ± 641 | 1427 |
| C₂₄/Dose | 1.04 ± 0.45 | 1.19 ± 0.73 | 2.02 ± 1.07 | 1.78 |
| (ng/mL/mg) | | | | |
| AUC₀₋₁₂ | 7.24 ± 0.32 | 16.3 ± 5.43 | 31.8 ± 10.43 | 40.5 |
| (μg · h/mL) | | | | |
| AUC₀₋₂₄ | 11.6 ± 0.154 | 25.8 ± 10.2 | 53.0 ± 15.71 | 65.0 |
| (μg · h/mL) | | | | |

C₂₄ = veliparib concentration at 24 hours post dosing
AUC₀₋₂₄ = area under the veliparib concentration-time curve from time 0 to 24 hours post dosing

TABLE 4
Veliparib Pharmacokinetic Parameters (Mean ±
SD) on Day 3 Following Twice-Daily (BID) Oral Administration
of Veliparib Extended Release Formulation C

| Pharmacokinetic | 200 mg | 400 mg | 600 mg |
|---|---|---|---|
| Parameters | (N = 4) | (N = 7) | (N = 4) |
| Tₘₐₓ (h) | 3.5 ± 2.5 | 3.1 ± 1.1 | 4.0 ± 1.6 |
| Cₘₐₓ (ng/mL) | 1351 ± 509 | 3121 ± 963 | 5185 ± 1176 |
| C₁₂ (ng/mL) | 651 ± 181 | 2117 ± 656 | 2875 ± 928 |
| C₁₂/Dose (ng/mL/mg) | 3.26 ± 0.91 | 5.29 ± 1.64 | 4.79 ± 1.55 |
| AUC₀₋₁₂ (μg · h/mL) | 11.7 ± 4.14 | 30.4 ± 11.8 | 49.8 ± 9.80 |

C₁₂ = veliparib concentration at 12 hours post dosing
AUC₀₋₁₂ = area under the veliparib concentration-time curve from time 0 to 12 hours post dosing

TABLE 5
Summary of Partial Response based on the Best Tumor Responses

| | ER | IR |
|---|---|---|
| | Parts 2 & 3 | Part 1 |
| Ovarian Cancer | | |
| BRCA mutated | 2/10 (20%) | 1/6 (17%) |
| BRCA wt | 2/5 (40%) | 0/6 (0%) |
| Breast Cancer | | |
| BRCA mutated | 4/6 (67%) | 1/6 (17%) |

Fifty-four subjects (39 with ovarian, fallopian tube, or peritoneal cancers, 14 with breast cancer, and 1 with prostate cancer) were included in an exposure-response analysis using nonlinear mixed-effects modeling approach with NONMEM software (version 7.3.0) (Table 6).

TABLE 6
Demographics of the Exposure-Response Analysis Dataset

| | Total | Part 1 | Part 2 | Part 3 |
|---|---|---|---|---|
| | N (%) | N (%) | N (%) | N (%) |
| Tumor Type | | | | |
| Ovarian/Fallopian | 39 (72.2) | 16 (66.7) | 15 (83.3) | 8 (66.7) |
| Tube/Peritoneal | | | | |
| Breast | 14 (25.9) | 7 (29.2) | 3 (16.7) | 4 (33.3) |
| Prostate | 1 (1.9) | 1 (4.2) | 0 (0.0) | 0 (0.0) |
| BRCA Status | | | | |
| Wild type | 12 (22.2) | 7 (29.2) | 5 (27.8) | 0 (0.0) |
| Mutated | 42 (77.8) | 17 (70.8) | 13 (72.2) | 12 (100) |

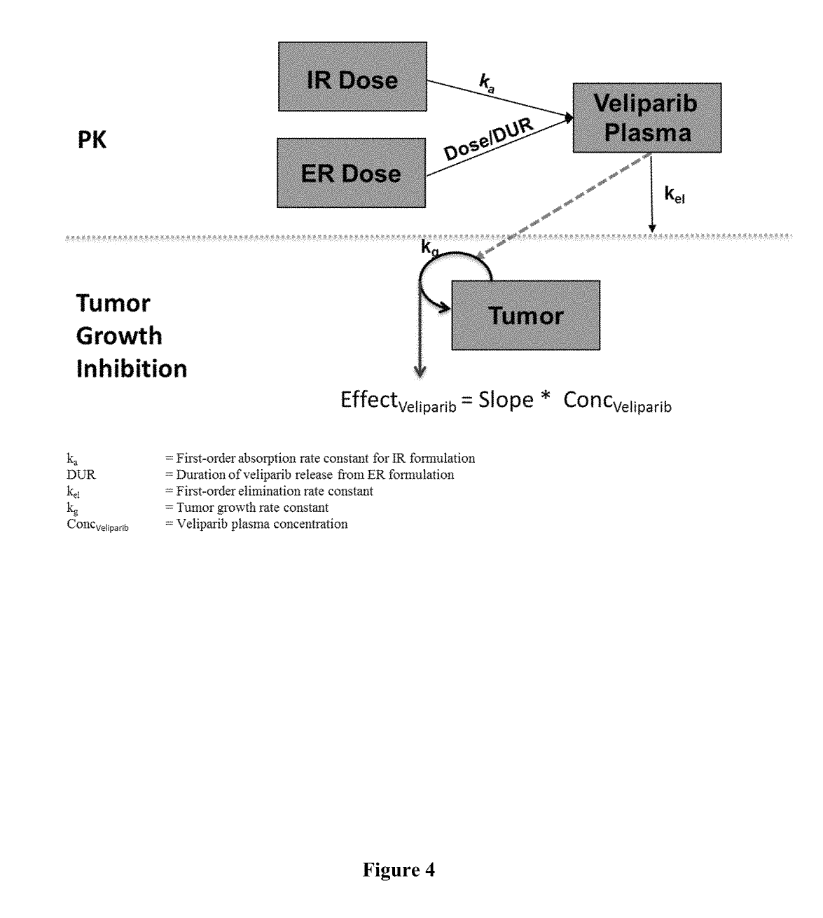

Veliparib pharmacokinetics (PK) was described with a one-compartment model with first-order elimination process (kₑₗ) (FIG. 4). For the IR formulation, absorption was modeled as a first-order process (kₐ) (Equations 1a and 1b). For the ER formulation, the absorption was modeled as a zero-order process (Dose/Duration) (Equations 2a and 2b).

Subsequently, the relationship between veliparib PK exposure and longitudinal tumor growth inhibition was analyzed. The time course of tumor growth inhibition was modeled using an exponential tumor growth model represented by a single tumor compartment linked to veliparib central compartment with linear inhibitory effect of veliparib on tumor growth (see FIG. 4 and Equation 3). The possible measurement error at the baseline was accounted for by allowing individual deviations from the observed baseline as between-subject variability. Inter-individual variability was incorporated on the slope and estimated baseline using exponential function and the residual variability was characterized using additive error. The influence of various covariates including formulation type, BRCA mutation status, and tumor type on the slope parameter was explored. Formulation type was introduced as a time-dependent covariate due to formulation switching during the study in some subjects.

$$\begin{matrix}\frac{dGut}{dt} = - {k}_{a} \times Gut & Equation 1 a \\ \frac{dConc}{dt} \times V / F = {k}_{a} \times Gut - {k}_{el} \times Conc \times V / F & Equation 1 b \\ \frac{dGut}{dt} = - \frac{Dose}{DUR} & Equation 2 a \\ \frac{dConc}{dt} \times V / F = \frac{Dose}{DUR} - {k}_{el} \times Conc \times V / F & Equation 2 b \\ \frac{dTumor}{dt} = ( {k}_{g} - Slope \times Conc ) \times Tumor & Equation 3\end{matrix}$$

The term “Gut” is the amount of veliparib remaining to be absorbed from the gut, the term “Cone” is veliparib plasma concentration, “V/F” represents veliparib volume of distribution, “kₐ” represents a first-order absorption rate constant, “DUR” represents duration of veliparib apparent release from the ER formulation, “kₑₗ” represents a first-order elimination rate constant, “k_{g}” represents a first-order tumor growth rate constant, and the term “Tumor” represents a sum of the longest diameter for all target lesions (mm).

The final tumor growth inhibition model included formulation type and BRCA status as covariates. Both covariates significantly reduced the objective function value when tested separately (p<0.05). The parameters of the final tumor growth inhibition model are presented in Table 8. All parameters were estimated with good precision. Exposure-response analyses showed that ER formulation had 2.94-fold (95% CI: 1.60, 5.42) greater slope compared to IR formulation suggesting higher efficacy of the ER formulation. Comparison of individual estimates of slope showed no apparent difference between different tumor types.

TABLE 8
Parameter Estimates of the Final Tumor
Growth Inhibition Model for Veliparib

| | | % Relative | |
|---|---|---|---|
| Parameter | Population | Standard | 95% Confidence |
| (Units) | Estimate | Error | Interval |
| Tumor growth rate | 0.00278 | 1.30 | (0.00166, 0.00470) |
| constant, kₐ (1/day) | | | |
| Baseline shift (mm) | 0.187 | 2.40 | (−4.28, 4.66) |
| Baseline correlation | 0.986 | 0.0381 | (0.910, 1.06) |
| coefficient | | | |
| Slope for IR and | 0.00102 | 2.02 | (0.000257, 0.00404) |
| BRCA-wt (L/(mg*day)) | | | |
| ER effect on Slope | 2.94 | 1.36 | (1.60, 5.42) |
| (i.e., ratio of Slope | | | |
| for ER to Slope for IR | | | |
| BRCA effect on Slope | 1.72 | 1.85 | (0.515, 5.73) |
| (i.e., ratio of Slope | | | |
| for BRCA-mut to Slope | | | |
| for BRCA-wt) | | | |

We claim:

1. An extended release (ER) oral pharmaceutical formulation of veliparib, or a pharmaceutically acceptable salt thereof.

2. The extended release (ER) oral pharmaceutical formulation of claim 1, wherein the dose-normalized C24 (concentration at 24 hours post dosing) of veliparib after once-daily administration of the formulation to a human subject is between about 1 and about 2 ng/mL/mg.

3. The extended release (ER) oral pharmaceutical formulation of claim 1, wherein the dose-normalized C12 (concentration at 12 hours post dosing) of veliparib after twice-daily administration of the formulation to a human subject is between about 3 and about 5 ng/mL/mg.

4. The extended release (ER) oral pharmaceutical formulation of claim 1, wherein the peak-to-trough concentration ratio is about 2 following twice-daily dosing.

5. The extended release (ER) oral pharmaceutical formulation of claim 1, wherein the peak-to-trough concentration ratio is about 4 following once-daily dosing.

6. The extended release (ER) oral pharmaceutical formulation of claim 1, wherein the release period is 6 to 16 hours.

7. A method of treating cancer comprising administering a therapeutically effective amount of an extended release (ER) oral pharmaceutical formulation of veliparib, or a pharmaceutically acceptable salt thereof.

8. The method of claim 7, wherein efficacy of the cancer treatment is increased by at least 50% as compared to an equivalent amount of an immediate release formulation of veliparib.

9. The method of claim 7, wherein efficacy of the cancer treatment is increased by at least 100% as compared to an equivalent amount of an immediate release formulation of veliparib.

10. The method of claim 7, wherein efficacy of the cancer treatment is increased by at least 200% as compared to an equivalent amount of an immediate release formulation of veliparib.

11. The method of claim 7, wherein efficacy of the cancer treatment is increased by at least 300% as compared to an equivalent amount of an immediate release formulation of veliparib.

12. The method of claim 7, wherein the dose-normalized C24 (concentration at 24 hours post dosing) of veliparib after once-daily administration of the ER formulation to a human subject is between about 1 and about 2 ng/mL/mg.

13. The method of claim 7, wherein the dose-normalized Cu (concentration at 12 hours post dosing) of veliparib after twice-daily administration of the ER formulation to a human subject is between about 3 and about 5 ng/mL/mg.

14. The method of claim 7, wherein the peak-to-trough concentration ratio of veliparib is about 2 following twice-daily dosing to a human subject.

15. The method of claim 7, wherein the peak-to-trough concentration ratio of veliparib is about 4 following once-daily dosing to a human subject.

16. The method of claim 7, wherein the release period of the extended release (ER) oral pharmaceutical formulation is 6 to 16 hours.

17. The method of any one of claim 7, wherein the cancer is selected from the group consisting of breast, ovarian, fallopian tube, primary peritoneal, and lung.
